(19) United States
(12) Patent Application Publication
(10) Pub. No.: US 2025/0090323 A1
(43) Pub. Date: Mar. 20, 2025

(54) ADJUSTABLE ANNULOPLASTY RING

(71) Applicant: EDWARDS LIFESCIENCES CORPORATION, Irvine, CA (US)

(72) Inventors: John Richard Carpenter, Irvine, CA (US); Sai Prasad Uppalapati, Plano, TX (US); Luke Anthony Zanetti, Parkton, MD (US); Ashley Nicolette Hinga, Eldersburg, MD (US); Stephen Epstein, Millersville, MS (US); Jyoti B Rao, Brea, CA (US)

(21) Appl. No.: 18/965,942

(22) Filed: Dec. 2, 2024

Related U.S. Application Data
(63) Continuation of application No. PCT/US2023/024196, filed on Jun. 1, 2023.
(60) Provisional application No. 63/365,758, filed on Jun. 2, 2022.

(51) Int. Cl. A61F 2/24

(52) U.S. Cl. CPC A61F 2/2448; A61F 2210/0009; A61F 2230/0034; A61F 2250/001

(57) ABSTRACT

An annuloplasty ring for repair of mitral valves whose shape can be altered directly or remotely to perform post-implant size and/or shape adjustments. One version of the annuloplasty ring has a size-adjustable remodeling inner core within an outer suture-permeable interface for attaching the ring to an annulus. The inner core has a size-adjustable segment formed by short spaced-apart tubular sections that can move toward and way from one another such as on rails. Alternatively, short sections separated by compressible connectors may be used. One or more tethers may be attached to one side of the size-adjustable segment and pulled from the other side to constrict the segment. Multiple tethers may be connect at different locations around the ring to provide some control over where the ring constricts. An adjustment handle with a flexible shaft may be operated from outside the body to adjust the ring post-implant.

### CROSS REFERENCE TO RELATED APPLICATIONS

The present application is a continuation of International Patent Application No. PCT/US23/24196, filed Jun. 1, 2023, which claims the benefit of U.S. Patent Application No. 63/365,758, filed Jun. 2, 2022, the entire disclosure which is incorporated by reference for all purposes.

### TECHNICAL FIELD

The present disclosure relates generally to annuloplasty rings, and in particular to an adjustable mitral annuloplasty ring and delivery system.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Various surgical techniques may be used to repair a diseased or damaged valve. A commonly used repair technique effective in treating incompetence is annuloplasty, which often involves reshaping or remodeling the annulus by attaching a prosthetic annuloplasty repair segment or ring thereto. The procedure is done with the heart stopped and the patient on cardiopulmonary bypass (“on pump”). For instance, the goal of a posterior mitral annulus repair is to bring the posterior mitral leaflet forward toward to the anterior leaflet to improve leaflet coaptation. Annuloplasty rings may be stiff, flexible or semi-rigid, and a “remodeling” annuloplasty ring typically has an inner core that is “generally rigid” or “semi-rigid” in that it will flex to a small extent but resist distortion when subjected to the stress imparted thereon by the mitral valve annulus of an operating human heart.

Currently, during a mitral valve repair procedure, the size of the annuloplasty ring is determined by comparing different sizer templates to the patient's anatomy until the surgeon determines which one looks correct based on, for example, anterior leaflet area or length, intercommissural distance, and so on. However, unlike for an aortic valve replacement, where the goal is to implant the largest valve that will safely fit the patient's anatomy, mitral repair procedures implant a repair device that is somewhat smaller than the annulus to reduce the perimeter, or, more importantly, the anterior-posterior (AP) diameter, of the valve and restore leaflet coaptation. The surgeon must make an “educated guess” as to how much reduction in size is appropriate for any given patient and their specific disease state. If the wrong size repair product is chosen, the result may be a poor outcome manifested by residual mitral regurgitation (MR), insufficient coaptation length, high pressure gradients, or systolic anterior motion (SAM). If any of these conditions are found once the patient is weaned off-pump, the surgeon must make the difficult decision of going back on pump, with its associated morbidity and mortality, or leaving the patient with a sub-optimal repair, and its associated sequalae.

Given the above challenges, it would be desirable to have an annuloplasty device that could be adjusted once the patient was weaned off-pump in order to fine-tune the AP diameter of the mitral valve in order to correct for small errors in the inherently imprecise sizing process. Such a ring would have the potential to reduce poor mitral valve repair outcomes and the need to go back on-pump in many cases. Once adjustments were made, the delivery system attachments could be disengaged, leaving the patient with a customized annuloplasty device that was tailored to their specific anatomy.

In attempts to vary the shape of the repair device, adjustable annuloplasty devices such as the CARDIOBAND® mitral and tricuspid reconstruction systems are available from Edwards Lifesciences Corp. of Irvine, CA. Other adjustable annuloplasty rings may be seen in U.S. Pat. Nos. 8,142,495, 8,349,002 and 9,107,749.

Modern annuloplasty rings such as the Edwards Physio II® annuloplasty ring have a very specific 3-dimensional shape, which has been shown to be important in maintaining and restoring anatomy as well as minimizing leaflet stresses. Adjustable devices have yet to successfully combine orifice downsizing with three-dimensional remodeling. Some are unduly complex while others have ring core elements that are completely rigid and do not appear to accommodate any shape change other than in the AP direction. Further, it is difficult to achieve optimal sizing of the mitral ring while the heart is on-pump; this sometimes leads to over- or under-sizing of the annulus, which may lead to post-operative complications.

Despite numerous designs presently available or proposed in the past, there is a need for an annuloplasty ring that may be shaped adjusted to effect repair of the malfunctioning valve while avoiding negative outcomes.

### SUMMARY

The application discloses an adjustable annuloplasty ring system that is surgically implanted on-pump, but can be slightly adjusted off-pump on the beating heart in order to optimize the annular size and reduce complications due to under- or over-sizing of the ring. The adjustable annuloplasty ring and a method for adjusting and locking the device is disclosed. The ring is surgically implanted like a normal annuloplasty ring, and then the patient closed up, the heart restarted, and a size adjustment made under visualization, if needed.

Disclosed here are mitral annuloplasty rings and shape adjustment systems for implant at a mitral annulus surrounding an anterior leaflet and a posterior leaflet. The mitral annulus is D-shaped with a straighter anterior aspect bordering an anterior leaflet opposite a more rounded posterior aspect bordering a posterior leaflet. The posterior leaflet defines P1, P2 and P3 regions in CCW sequence looking from the atrial side.

A first exemplary system comprises an annuloplasty ring defining a peripheral shape around a central aperture, the peripheral shape being D-shaped with a straighter anterior side adapted to be implanted adjacent the anterior leaflet opposite a more rounded posterior side adapted to be implanted adjacent the posterior leaflet. The annuloplasty ring has an inner core and a suture-permeable interface surrounding the inner core and extending around the peripheral shape. The inner core has multiple arcuate segments on the posterior side that are linearly or circumferentially compressible to enable constriction of the ring while the anterior side is not size-adjustable. The system further includes a proximal control handle having a flexible shaft extending therefrom, a distal end of the shaft being adapted to engage an access port in the ring. At least two tension filaments extend from the control handle through the flexible shaft and access port and extend around an inner core of the ring to separate anchor points, wherein an actuator on the control handle is configured to apply tension to the filaments and constrict the inner core by bringing at least two of the arcuate segments closer together.

In the first system, the peripheral shape may be open and defines two free ends, and the access port may be located at one of the free ends, or on an atrial side of the ring spaced from one of the free ends. The actuator is preferably a rotatable ring.

The anchor points may be located so that arcuate segments adjacent at least the P2 and P3 regions of the posterior leaflet are independently adjustable. Or there are three tension filaments extending around the inner core of the ring to different anchor points, and there is one actuator on the control handle for each tension filament. With three filaments, the anchor points may be located so that arcuate segments adjacent the P1, P2 and P3 regions of the posterior leaflet are independently adjustable.

The arcuate segments may comprise short core members that slide relative to curved struts passing through apertures therein, or the arcuate segments comprise short core members separated by linearly or circumferentially compressible sections and both made of silicone.

A second exemplary system comprises an annuloplasty ring defining a peripheral shape around a central aperture, the peripheral shape being D-shaped with a straighter anterior side adapted to be implanted adjacent the anterior leaflet opposite a more rounded posterior side adapted to be implanted adjacent the posterior leaflet. The annuloplasty ring has an inner core and a suture-permeable interface surrounding the inner core and extending around the peripheral shape. The inner core has a size-adjustable segment adjacent just the P2-P3 regions of the posterior leaflet that is linearly or circumferentially compressible to enable constriction of the ring while the P1 region and anterior side are not size-adjustable. The system further includes a proximal control handle having a flexible shaft extending therefrom, a distal end of the shaft being adapted to engage an access port in the ring. At least one tension filament extends from the control handle through the flexible shaft and access port and extends around an inner core of the ring to an anchor point. An actuator on the control handle is configured to apply tension to the filament and constrict the size-adjustable segment of the inner core.

In the second system, the peripheral shape may be open and defines two free ends, and the access port may be located at one of the free ends, or on an atrial side of the ring spaced from one of the free ends. The actuator is preferably a rotatable ring.

There may be three tension filaments extending around the inner core of the ring to different anchor points, and is one actuator on the control handle for each tension filament. The size-adjustable segment may comprise short core members that slide relative to curved struts passing through apertures therein, or short core members separated by linearly compressible sections, both made of silicone.

A further understanding of the nature and advantages will become apparent by reference to the remaining portions of the specification and drawings.

### BRIEF DESCRIPTION OF THE DRAWINGS

Features and advantages of the present disclosure will become appreciated as the same become better understood with reference to the specification, claims, and appended drawings wherein:

FIG. 1 is an atrial plan view of a mitral valve and leaflets indicating common nomenclature initials for regular anatomical features;

FIG. 2 is a schematic view of the three-dimensional shape of the mitral annulus with several anatomical landmarks indicated;

FIGS. 3A-3E are orthogonal views of an exemplary annuloplasty ring of the present application;

FIG. 4A is an atrial plan view of an open or C-shaped annuloplasty ring having a size-adjustable segment in a posterior area thereof, and FIG. 4B is a plan view of the annuloplasty ring after cinching to reduce the size of the adjustable segment;

FIG. 5 is a schematic view of the annuloplasty ring in FIG. 4A after implant and connected to a size-adjustment mechanism/delivery system that extends through the left atrium and out of the body;

FIG. 6 is an atrial plan view of an alternative open or C-shaped annuloplasty ring having size-adjustable segments around the majority of a posterior area thereof;

FIG. 7 is a schematic view of a size-adjustment mechanism/delivery system engaging the annuloplasty ring of FIG. 6;

FIG. 8A is an atrial plan view of another open or C-shaped annuloplasty ring having a size-adjustable segment in a posterior area thereof, and FIG. 8B is a plan view of the annuloplasty ring after cinching to reduce the size of the adjustable segment;

FIG. 9 is a schematic view of the annuloplasty ring in FIG. 8A after implant and connected to a size-adjustment mechanism/delivery system that extends through the left atrium and out of the body;

FIG. 10 is an atrial plan view of a closed or D-shaped annuloplasty ring having a size-adjustable posterior segment, and FIG. 11 is a schematic view of a size-adjustment mechanism/delivery system engaging the annuloplasty ring;

FIG. 12A is an atrial plan view of another closed or D-shaped annuloplasty ring having a size-adjustable posterior segment, and FIG. 12B is a plan view of the annuloplasty ring with an outer suture interface shown in dashed lines to illustrate an adjustable inner core thereof;

FIG. 13 is a schematic view of the annuloplasty ring in FIG. 12A after implant and connected to a size-adjustment mechanism/delivery system that extends through the left atrium and out of the body; and

FIG. 14 is a perspective view of another closed or D-shaped annuloplasty ring having a size-adjustable posterior segment, and FIG. 14A is an enlargement of a cross-section thereof.

### DETAILED DESCRIPTION

The right ventricle RV and left ventricle LV are separated from the right atrium RA and left atrium LA, respectively, by the tricuspid valve TV and mitral valve MV; e.g., the atrioventricular valves. Though correction of the mitral annulus is the primary focus of the present application, it should be understood that certain characteristics of the annuloplasty rings described herein may equally be used to treat the tricuspid valve TV, and thus the claims should not be constrained to the mitral ring unless expressly limited.

The term “axis” in reference to the illustrated annuloplasty rings, and other non-circular or non-planar rings, refers to a line generally through the centroid of the ring periphery when viewed in plan view. “Axial” or the direction of the “axis” can also be viewed as being parallel to the average direction of blood flow within the valve orifice and thus within the ring when implanted therein. Stated another way, an implanted mitral ring orients about a central flow axis aligned along an average direction of blood flow through the mitral annulus from the left atrium to the left ventricle. The plan views of the annuloplasty rings illustrated herein are as looking from the atrial side in the direction of blood flow. For the purpose of orientation, therefore, the atrial side of the ring is up in the ventricular site is down.

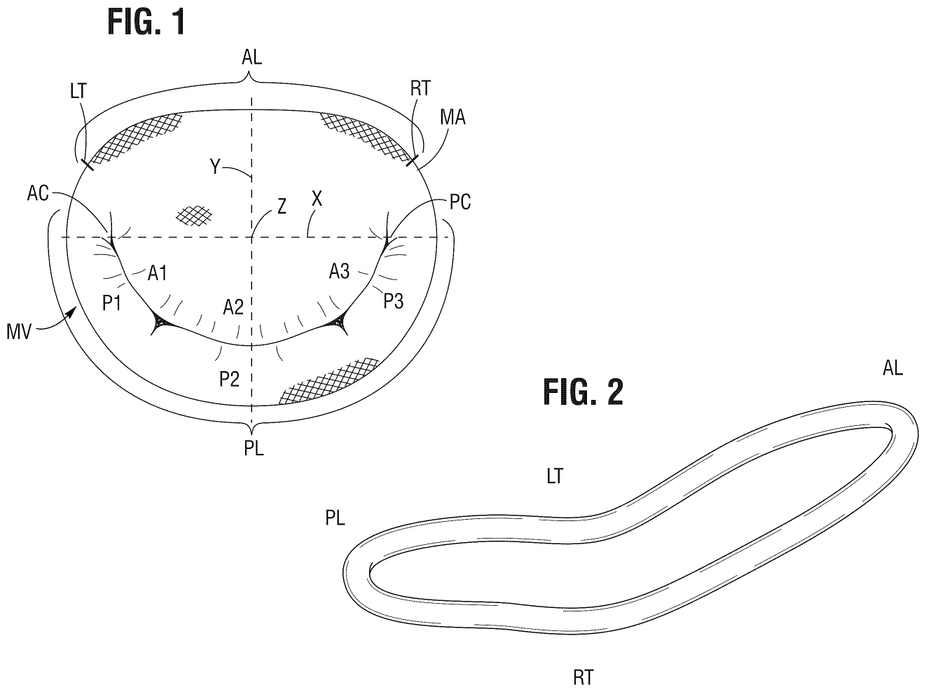

FIG. 1 is a schematic perspective view from the atrial side of a mitral valve MV with posterior being down and anterior being up. The mitral valve MV primarily comprises a pair of coapting leaflets—an anterior leaflet AL and a posterior leaflet PL—secured around their outer edges to a fibrous mitral annulus MA. The surrounding mitral annulus MA is often described as D-shaped with a somewhat straighter side adjacent the anterior leaflet AL and a more rounded or convex side adjacent the posterior leaflet PL. The mitral annulus MA is typically viewed as having a major axis X that intersects both the first and third posterior scallops P1 and P3, approximately at the commissures AC, PC, and a minor axis Y that intersects and generally bisects the middle posterior scallop P2. A central flow axis Z is arbitrarily defined at the intersection of the major and minor axes X, Y.

The leaflets are shaped such that the line of coaptation resembles a smile that approximately parallels the posterior aspect of the mitral annulus MA. The anterior leaflet AL spans a smaller peripheral aspect around the mitral annulus MA than the posterior leaflet PL, but the anterior leaflet AL has a convex free edge that extends farther into the orifice defined by the mitral annulus MA. The posterior leaflet PL, on the other hand, has a generally concave free edge. Two commissures—an anterior commissure AC and a posterior commissure PC-generally defined the intersection of the line of coaptation between the two leaflets AL, PL and the mitral annulus MA. The posterior leaflet is divided into three scallops or cusps, sometimes identified as P1, P2, and P3, starting from the anterior commissure AC and continuing in a counterclockwise direction to the posterior commissure PC. Per convention, a major axis of the mitral annulus intersects both the first and third posterior scallops P1 and P3, approximately at the commissures AC, PC, and a minor axis intersects and generally bisects the middle posterior scallop P2. The anterior leaflet also features scallops or regions labeled A1, A2, and A3 as indicated in FIG. 1.

As illustrated, the mitral annulus has a kidney or rounded D-shape around its periphery. The mitral anterior leaflet AL attaches to a somewhat straight anterior fibrous portion of the mitral annulus, which makes up about one-third of the total mitral annulus circumference. The anterior fibrous annulus, the two ends of which are called the fibrous left and right trigones LT, RT, forms part of the central fibrous skeleton of the heart. The arcuate muscular portion of the mitral annulus constitutes the remainder of the mitral annulus, and the posterior leaflet PL attaches thereto. The anterior commissure AC and the posterior commissure PC are located just posterior to each fibrous trigone.

FIG. 2 is a schematic view of the three-dimensional shape of the mitral annulus with several anatomical landmarks indicated. In particular, the anterior leaflet AL is separated from the posterior leaflet PL by the left and right trigones LT, RT. The three-dimensional shape is somewhat like a saddle, with the trigones LT, RT in a valley and the leaflets AL, PL rising up.

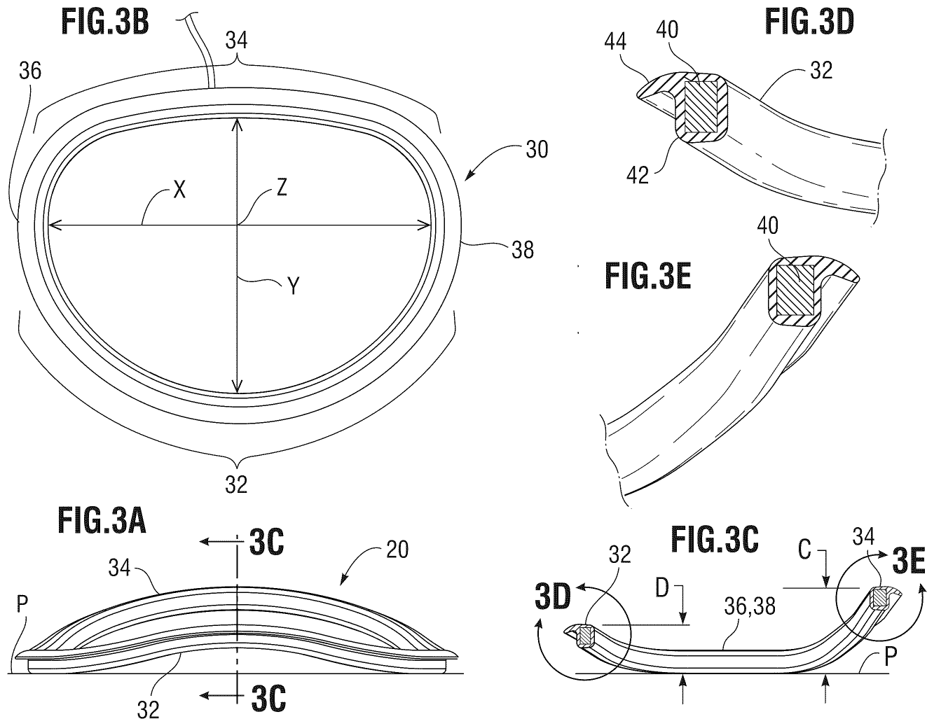

As seen in FIGS. 3A-3C, the exemplary annuloplasty ring 30 has a posterior portion 32 opposite an anterior portion 34, with side segments 36, 38 in between. Some nomenclature has the posterior portion 32 extending roughly around the posterior leaflet PL (FIG. 2), extending between the leaflet commissures AC, PC, but generally the posterior portion 32 is bisected by the minor axis Y and extends at least around the middle posterior scallop P2 of the posterior leaflet PL. A major axis X and a minor axis Y are indicated which, when the annuloplasty ring 30 is implanted, coincide with the same axes of the native mitral annulus, such that blood will flow into the page generally parallel to a flow axis Z through the middle of the ring. The plan view shape of the annuloplasty ring 30 is kidney or rounded D-shaped so as to conform to the peripheral shape of the typical mitral annulus.

The annuloplasty ring 30 may be three-dimensional with an upward bow in the posterior portion 32 as well as an upward bow in the anterior portion 34, as seen in FIGS. 3A and 3C. Preferably the anterior portion 34 bows upward a distance C from a reference plane P more than an upward bow D of the anterior portion 34. The side segments 36, 38 may lie in the reference plane P such that the ring 30 is partially planar with the two opposite upward bows, to form somewhat of a saddle shape so as to better conform to the native mitral annulus, as depicted in FIG. 2. The shape of the ring 30 is similar to that of the Physio II® annuloplasty ring available from Edwards Lifesciences of Irvine, CA.

FIGS. 3D and 3E are enlarged sectional views of the annuloplasty ring 30 as seen in section in FIG. 3C. In a preferred example, the ring construction includes an adjustable inner core 40, as will be described, and surrounded by a suture-permeable interface. In the illustrated example, the inner core 40 is shown as solid and rectangular, with a greater axial dimension than radial dimension, though other configurations are possible. As will be explained below, the material of the inner core 40 is an elastic-plastic material such as stainless steel or titanium alloy, or an elastic plastic metal, an elastic-plastic polymer such as nylon (polyamide), polyacetal (e.g., DELRIN® polyoxymethylene), or the like may also be utilized. The annuloplasty ring 30 is thus a remodeling ring which, when adjusted in size, retains its general shape against the forces associated with normal cycling of the heart.

The suture-permeable interface may include an elastomeric sleeve 42 closely surrounding the core and a fabric outer cover (not shown), for example, a polyethylene terephthalate (PET) fabric cover. In the preferred example the elastomeric sleeve 42, which may be silicone rubber, is generally tubular and molded to have a radially outwardly-extending flange 44 to facilitate suturing of the ring 30 to the mitral annulus. The ring 30 may be secured with sutures, staples, or other such devices to an inside fibrous ledge of the mitral annulus. In a typical procedure, the surgeon anchors an array of sutures through the annulus and then threads them through corresponding locations around the interface on the outside of the ring 30. The ring is parachuted down the suture array to be seated at the annulus before tying off the sutures.

In Degenerative Mitral Regurgitation, a primary cause for the onset of regurgitation is the dilation of the MV annulus. Typically the dilation of the annulus is localized in the P2 to P3 region. The need is to reshape the annulus to an appropriate size, to support the annulus in the surgically remodeled shape, and to reduce the time for implementation of the reinforcement ring/band.

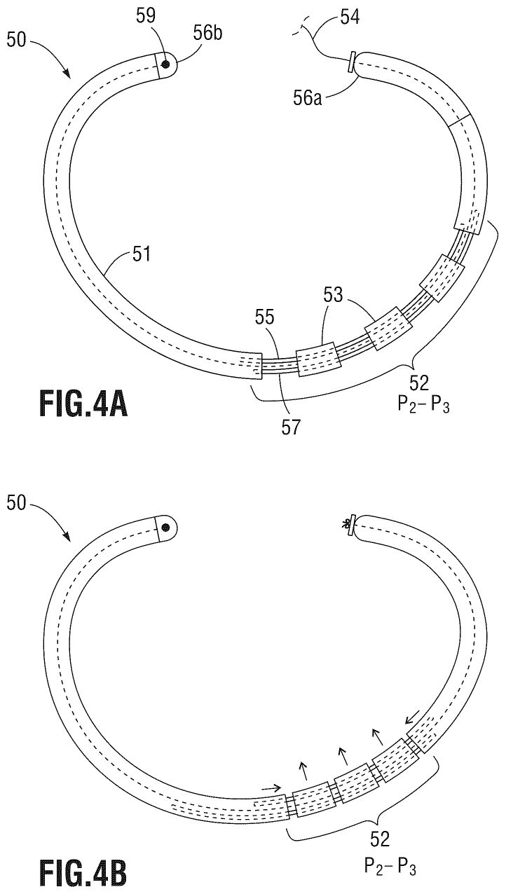

FIG. 4A is an atrial plan view of an open or C-shaped annuloplasty ring 50 having a core with a size-adjustable segment 52 in a posterior area, in particular the P2 to P3 region, of the annulus, and FIG. 4B is a plan view of the annuloplasty ring after cinching to reduce the size of the adjustable segment (arrows). The size-adjustable segment 52 comprises multiple short posterior arcuate segments 53 that are capable of constricting toward each other. In this way, and by using a cinch wire 54 inserted through one free end 56a of the ring 50, the ring can be sized exactly to the required shape. The cinch wire 54 may extend around the entire periphery of the interior of the ring 50 through the arcuate segments 53 to a second free end 56b, where it anchors such as at crimp or knot 59. The arcuate segments 53 may slide over a curved strut 55 which is anchored into the core 51 on one side of the size-adjustable segment 52. A second curved strut 57 over which the arcuate segments 53 slide may be embedded into the core 51 on the other side of the size-adjustable segment 52 for stability. By cinching at the P2 to P3 region of the annulus, and not in the P1 region, correction to the actual dilated area is possible. In addition, this cinching may also eliminate the need to create a plication in the annulus. (Note: The necessity to apply a plication introduces increased operative risk, increased surgical skill required to create a plication, and increased time to apply the plication to the annulus.)

The cinch wire 54 may alternatively be connected to the curved strut 55 passing through the arcuate segments 53. The arcuate segments 53 slide over the curved strut 55 which is anchored into the core 51 on the side of the size-adjustable segment 52 opposite the free end 56a. The second curved strut 57 over which the arcuate segments 53 slide may be embedded into the core 51 on the other side of the size-adjustable segment 52 for stability.

The sizing of the ring 50 can be done using conventional methods, then during the application, cinched to optimize the repair. The ability to cinch the annulus in the P2-P3 region provides greater stability to the repair in this weakened location.

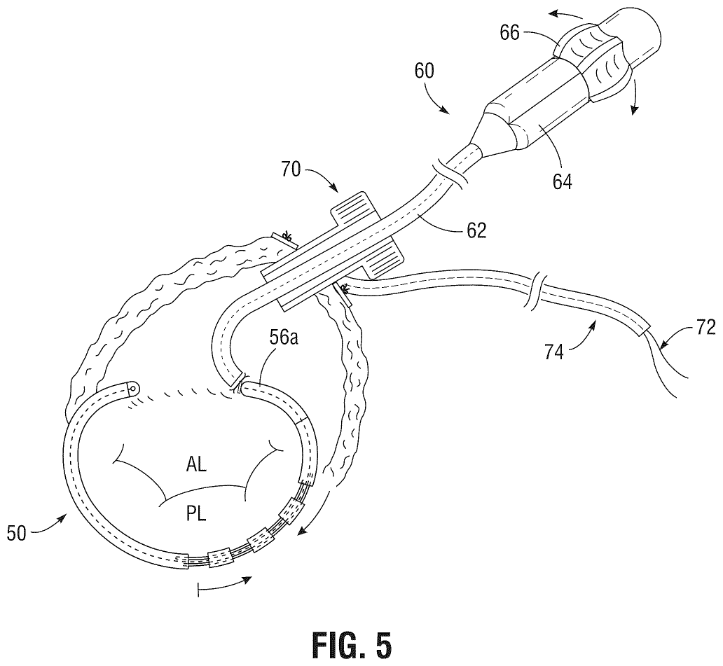

FIG. 5 is a schematic view of the annuloplasty ring 50 in FIG. 4A after implant anchored by sutures or clips to the annulus) and connected to a size-adjustment mechanism/delivery system 60 that extends through the left atrium and out of the body. The delivery system 60 has an elongated, flexible sheath 62 that engages the first free end 56a of the ring 50, and extends out of the body to a proximal handle 64. A rotating dial 66 or other such actuator is connected to pull or create a tension on the cinch wire 54 to reduce the size of the annuloplasty ring 50. The sheath 62 may pass through an introducer 70 having internal hemostatic valve therein. The introducer 70, in turn, extends through a puncture in the left atrial wall which may be tightened around the sheath 62 using a pledgeted purse-string suture 72 passing through a purse-string tourniquet 74. The size-adjustable segment 52 may then be adjusted inward to pull one side of the posterior leaflet PL toward the anterior leaflet AL.

Placement of the annuloplasty ring 50 is typically done on a stopped, flaccid heart, during which time it is difficult to fully evaluate the effectiveness of the ring/band in properly sizing the effective orifice of the MV, and to also optimize the coaptation of the leaflets to reduce or eliminate MR. Consequently, the final adjustment and optimization of the adjustable annuloplasty ring 50 can be accomplished on a beating heart with the delivery system 60, using echo guidance to optimize the ring adjustment. Sizing of the ring would follow conventional methods, being that the adjustable ring can be optimized a few millimeters in either direction. Once the optimization is complete, the ring 50 can be locked, the cinch wire 54 and delivery system 60 removed, and finally the hemostatic introducer 70 removed while simultaneously tightening the purse-string suture 72 to minimize bleeding and allow for final closure of the atrial wall incision.

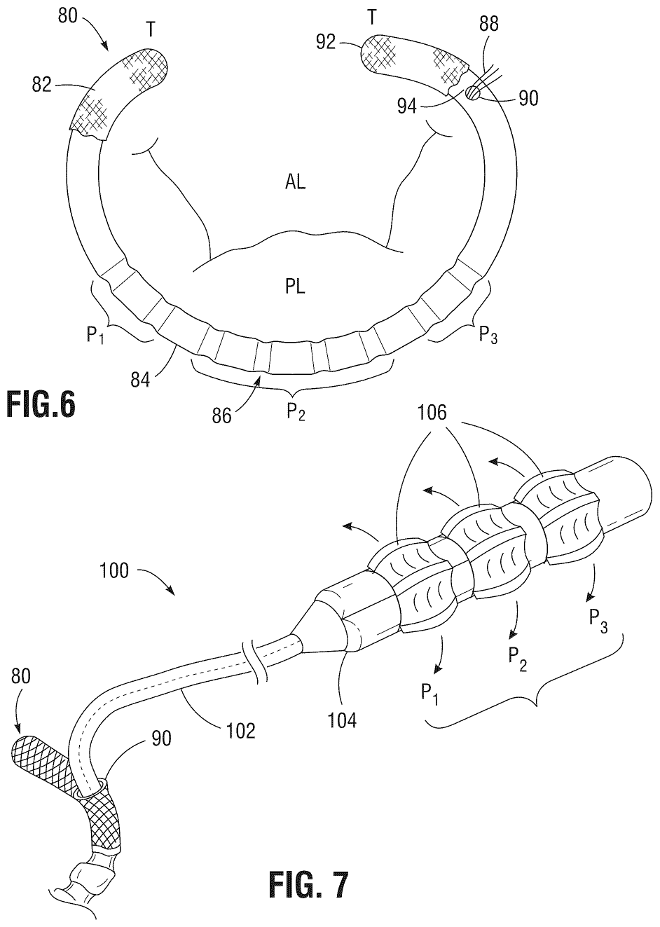

FIG. 6 is an atrial plan view of an alternative open or C-shaped annuloplasty ring 80 having size-adjustable segments around the majority of the posterior area thereof. The annuloplasty ring 80 is somewhat similar to the ring 50 described above, but also incorporates adjustability of the P1 region to correct prolapse in the P1 region post atrial closure. No adjustment in the anterior region is typically needed due to the more fibrous nature of the annulus at that location. Specifically, the annuloplasty ring 80 has an outer textile cover 82 surrounding an inner core 84 at least partly made of linearly or circumferentially compressible elastomeric (e.g., silicone) segments 86. The core 84 has a hollow interior lumen through which passes a plurality (e.g., three) tension filaments 88.

The tension filaments 88 emerge from the inner lumen at a port 90 located on an atrial side adjacent one free end 92 of the ring 80. Each tension filament 88 extends around the ring 80 a predetermined distance where it is secured to the core 84. Preferably, each tension filament 88 extends through a cable sheath which terminates short of the anchor point for the filament, such that tension on the filament compresses a discrete segment of the ring 80. For instance, one of the tension filaments 88 may extend all the way clockwise (CW) around the ring 80 to an anchor point at the distal end of the P1 segment. The associated cable sheath terminates and is secured to the core 84 at a proximal end of the P1 segment, so that tension on the filament compresses just the P1 segment independently. The same may be done independently with two other filaments 88 for the P2 and P3 segments to provide separate size adjustments of each of the three segments. In this way, greater size reduction is achievable, as well as the ability to adjust the band to correct for coaptation issues, and upsize/downsize the ring 80 in specific regions of the mitral annulus.

In addition to size adjustability in multiple discrete segments, the ring 80 has a flexible joint 94 at a posterior commissural marker to reduce the stress on the sutures/tissue in the portion of the ring which extends from the posterior commissural marker toward the posterior trigone. This is a location where dehiscence may occur due to excessive stresses. Positioning the port 90 on the ring 80 where adjustments are made allows the device to pass through the existing surgical incision without the need for additional insertion sites to be established. Closure of the wound in this location, following the removal of a tensioning device, can be accomplished with a running atrial closure suture. This location also has the advantage of not putting additional loads on the MV trigone during adjustment which may injure the MV.

FIG. 7 is a schematic view of a size-adjustment mechanism/delivery system 100 engaging the annuloplasty ring 80 of FIG. 6. The delivery system 100 has an elongated, flexible sheath 102 that engages the port 90 of the ring 80, and extends through the left atrium and out of the body through an access incision to a proximal handle 104. A plurality (e.g., three) rotating dials 106 or other such actuators are connected to pull or create a tension on the tension filaments 88 to reduce the size of different segments of the annuloplasty ring 80. The sheath 102 may pass through an introducer 70 having an internal hemostatic valve, such as described above for FIG. 5, which in turn passes through a puncture in the left atrial wall which may be tightened around the sheath 62 using a pledgeted purse-string suture 72 passing through a purse-string tourniquet 74, also described above.

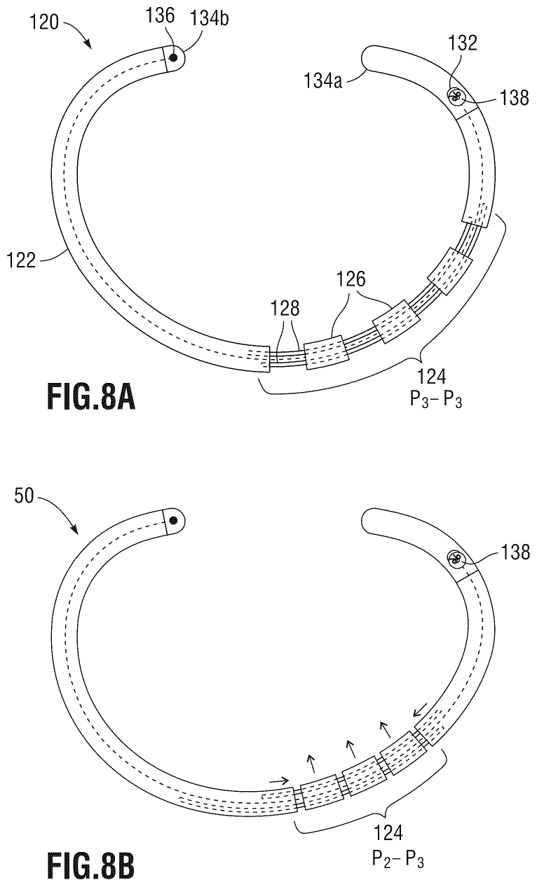

FIG. 8A is an atrial plan view of still another open or C-shaped annuloplasty ring 120 having a size-adjustable segment in just the P2-P3 region thereof, and FIG. 8B is a plan view of the annuloplasty ring after cinching to reduce the size of the adjustable segment. Again, no adjustment in the anterior region is needed, and none in the P1 region either. The ring 120 may be constructed as described elsewhere herein, with an outer textile cover (not shown) surrounding an inner core 122 at least partly made of a compressible segment 124. The compressible segment 124 may be elastomeric (e.g., silicone), such as seen at 86 in FIG. 6, or be telescoped or otherwise accordioned so as to be linearly or circumferentially compressible. For instance, several short core members 126 may be held in alignment by one or more inner curved struts 128. The struts 128 may pass through bores in the core members 126 with sliding movement provided therebetween.

The core 122 and core members 126 has a hollow interior lumen through which passes a tension wire or filament 130. The filament 130 enters through a port 132 on an atrial side near one free end 134a of the ring 120 and extends to an anchor point 136 located beyond a midpoint of the ring 120; i.e., anywhere from a midpoint of the P2 segment around to second free end 134b. Pulling tension on the filament 130 pulls the anchor point 136 in a counter-clockwise (CCW) direction and causes the P2-P3 segment to compress linearly or circumferentially by virtue of the core members 126 sliding over the struts 128.

FIG. 8B shows the ring 120 after the P2-P3 segment has been linearly or circumferentially compressed or collapsed. After compression to the desired degree, a tourniquet clip 138 may be installed at the port 132 to lock the tension filament 130 in place. The clip 138 may be made of a material such as cobalt-chromium in order to hold well even at low temperatures of the tissue during the open heart procedures. For instance, a clip such as disclosed in U.S. Pat. No. 9,498,202 may be used, the contents of which are expressly incorporated herein.

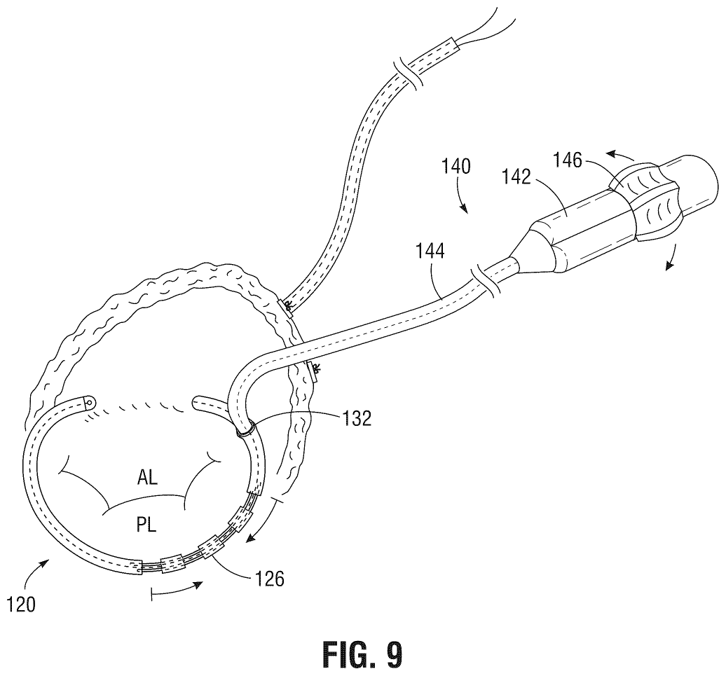

FIG. 9 is a schematic view of the annuloplasty ring 120 in FIG. 8A after implant and connected to a size-adjustment mechanism/delivery system 140 that extends out of the body. As described above, the delivery system 140 has a proximal handle 142 from which a flexible shaft or sheath 144 extends distally into the body to the access port 132. A size-adjustment actuator 146 such as a dial as shown is built into the handle 142 for adjusting the size of the ring 120 from outside the body and while the heart beats. The size-adjustable segment 124 may then be adjusted inward using the actuator dial 146 to pull one side of the posterior leaflet PL toward the anterior leaflet AL.

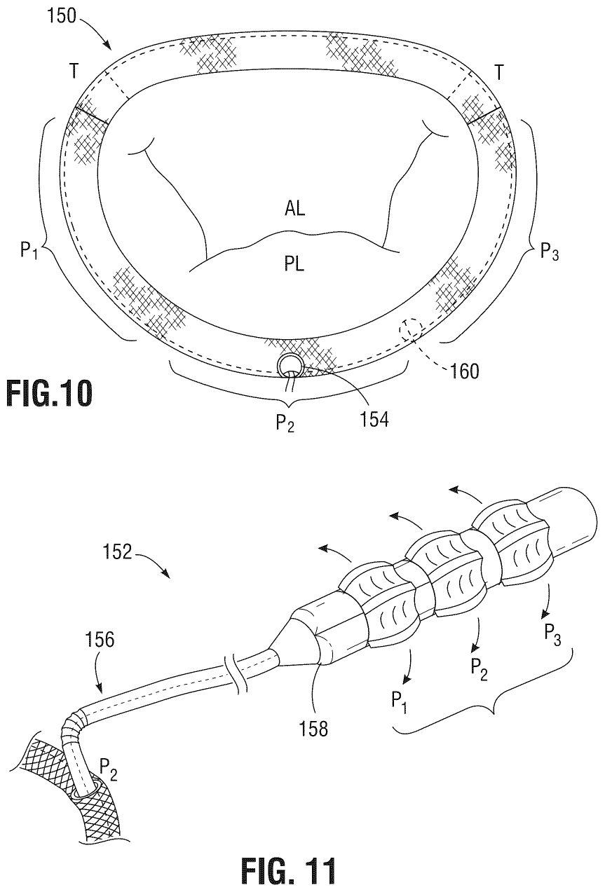

FIG. 10 is an atrial plan view of a closed or D-shaped annuloplasty ring 150 having a size-adjustable posterior segment, and FIG. 11 is a schematic view of a size-adjustment mechanism/delivery system 152 engaging the annuloplasty ring. In the closed ring 150, an access port 154 is provided to which a distal end of a flexible shaft 156 of the delivery system 152 is removably attached. The access port 154 is located central to the P2 segment. The delivery system 152 has a proximal handle 158 with a plurality of adjustment dials 158 for different segments of the ring 150. For instance, multiple cable sheaths (not shown) may extend from the handle 158 through the shaft 156 and into the port 154. Each cable sheath then extends around inside of a ring core to a particular location, from which a tension filament extends to a farther anchor point. Pulling the tension filaments enables size adjustment of various segments in the ring 150. For instance, each of the P1 and P3 regions may be separately adjusted. By moving the access port 154 to a location between the P1 and P2 regions, such as indicated in dashed line 160, adjustment of the P2 region independently may also be done.

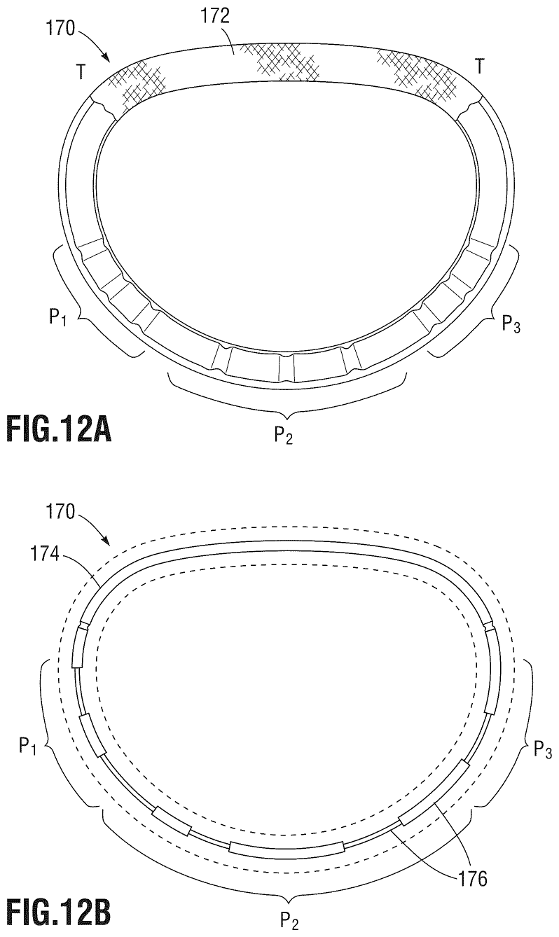

FIG. 12A is an atrial plan view of another closed or D-shaped annuloplasty ring 170 having a size-adjustable posterior segment, and FIG. 12B is a plan view of the annuloplasty ring with an outer suture interface 172 shown in dashed lines to illustrate an adjustable inner core 174 thereof. The outer cover or suture interface 172 may be a tube of flexible material such as silicone or polypropylene, and a fabric outer. The inner core 174 of FIG. 12B comprises a number of telescoped sections 176 in series which, together, enable linear or circumferential compression of the posterior aspect of the ring 170. The telescoped sections 176 may extend around from trigone location to trigone location as shown, approximately spanning three-quarters of the ring circumference. An anterior aspect is solid and non-adjustable.

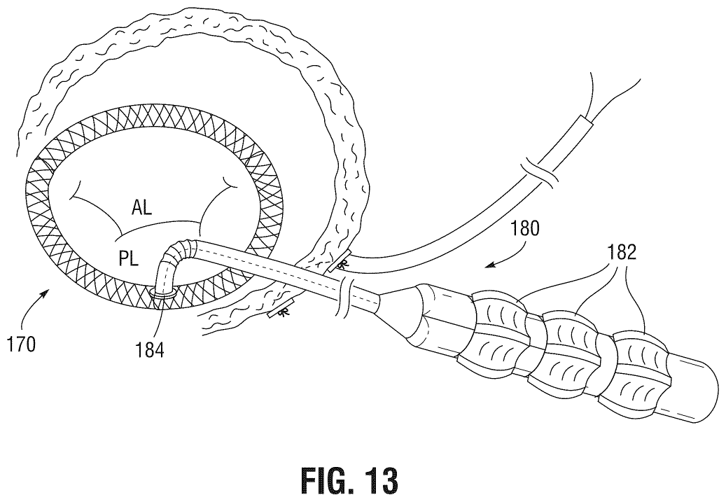

FIG. 13 is a schematic view of the annuloplasty ring 170 in FIG. 12A after implant and connected to a size-adjustment mechanism/delivery system 180 that extends out of the body. As before, actuators 182 on a proximal handle are connected via tension filaments (not shown) to various telescoped sections 176 within the ring 170 to enable constriction of different segments of the ring after implant and under visualization. The sheath of the delivery system 180 may engage an access point 184 on a central posterior location of the ring 170, or in various other locations as described herein.

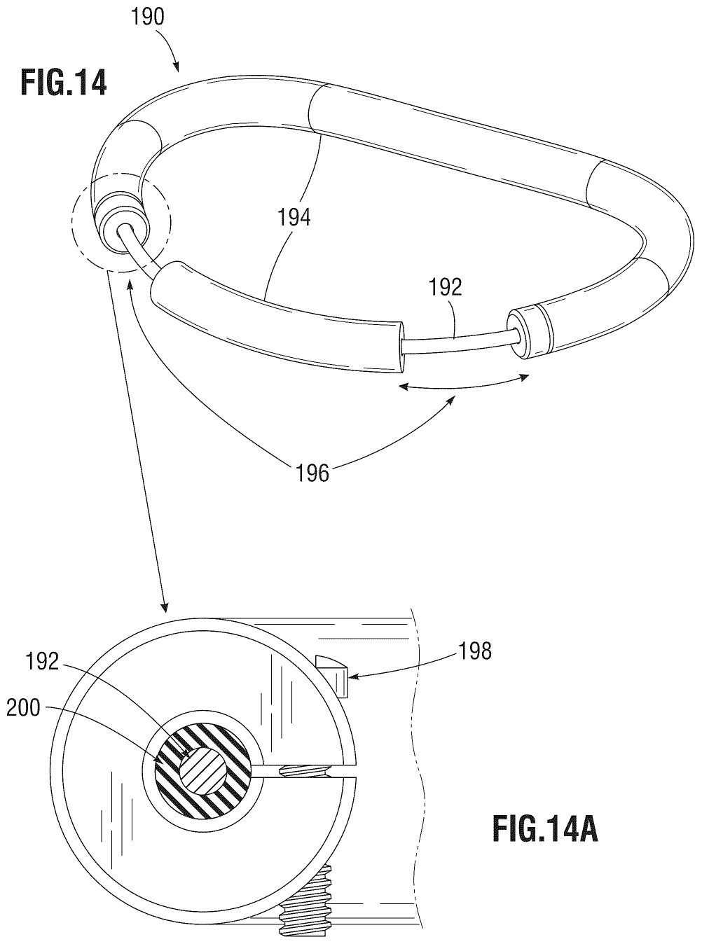

FIG. 14 is a perspective view of another closed or D-shaped annuloplasty ring 190 having a size-adjustable posterior segment, and FIG. 14A is an enlargement of a cross-section thereof. The annuloplasty ring 190 comprises a core wire 192 surrounded in part by a ring body 194 in segments. There are two telescoping regions 196 of exposed wire. The regions 196 of exposed wire can expand on contract to the desired ring size, and then may be locked in by tightening a screw 198 to clamp the wire in place. A sliding or expanding sleeve 200 of an elastomer such as rubber, for example, could be used to apply a frictional holding force to the wire core 192 to avoid premature constriction. The ring body segments 194 may be pulled together over the wire 192 using tethers or filaments as explained elsewhere herein. Namely, using an access point at the center of the posterior segment, two filaments may extend along or through wire core 192 and anchor to the remainder of the core. Or one or more filaments may extend from one trigone region around the ring interior to anchor points which pull one or more segments to constrict the ring.

While the foregoing is a complete description of the preferred examples, various alternatives, modifications, and equivalents may be used. Moreover, it will be obvious that certain other modifications may be practiced within the scope of the appended claims.

What is claimed is:

1. A mitral annuloplasty ring and shape adjustment system for implant at a mitral annulus surrounding an anterior leaflet and a posterior leaflet, the mitral annulus being D-shaped with a straighter anterior aspect bordering an anterior leaflet opposite a more rounded posterior aspect bordering a posterior leaflet, and the posterior leaflet defining P1, P2 and P3 regions in CCW sequence looking from the atrial side, comprising:
an annuloplasty ring defining a peripheral shape around a central aperture, the peripheral shape being D-shaped with a straighter anterior side adapted to be implanted adjacent the anterior leaflet opposite a more rounded posterior side adapted to be implanted adjacent the posterior leaflet, the annuloplasty ring having an inner core and a suture-permeable interface surrounding the inner core and extending around the peripheral shape, the inner core having multiple arcuate segments on the posterior side that are linearly or circumferentially compressible to enable constriction of the ring while the anterior side is not size-adjustable, the system including a proximal control handle having a flexible shaft extending therefrom, a distal end of the shaft being adapted to engage an access port in the ring, and at least two tension filaments extending from the control handle through the flexible shaft and access port and extending around an inner core of the ring to separate anchor points, wherein an actuator on the control handle is configured to apply tension to the filaments and constrict the inner core by bringing at least two of the arcuate segments closer together.

2. The system of claim 1, wherein the peripheral shape is open and defines two free ends.

3. The system of claim 1, wherein the access port is located at one of the free ends.

4. The system of claim 1, wherein the access port is located on an atrial side of the ring spaced from one of the free ends.

5. The system of claim 1, wherein the actuator is a rotatable ring.

6. The system of claim 1, wherein there are three tension filaments extending around the inner core of the ring to different anchor points, and there is one actuator on the control handle for each tension filament.

7. The system of claim 1, wherein the anchor points are located so that arcuate segments adjacent at least the P2 and P3 regions of the posterior leaflet are independently adjustable.

8. The system of claim 1, wherein there are three tension filaments extending around the inner core of the ring to different anchor points, and there is one actuator on the control handle for each tension filament.

9. The system of claim 8, wherein the anchor points are located so that arcuate segments adjacent the P1, P2 and P3 regions of the posterior leaflet are independently adjustable.

10. The system of claim 1, wherein the arcuate segments comprise short core members that slide relative to curved struts passing through apertures therein.

11. The system of claim 1, wherein the arcuate segments comprise short core members separated by linearly or circumferentially compressible sections and both made of silicone.

12. A mitral annuloplasty ring and shape adjustment system for implant at a mitral annulus surrounding an anterior leaflet and a posterior leaflet, the mitral annulus being D-shaped with a straighter anterior aspect bordering an anterior leaflet opposite a more rounded posterior aspect bordering a posterior leaflet, and the posterior leaflet defining P1, P2 and P3 regions in CCW sequence looking from the atrial side, comprising:
an annuloplasty ring defining a peripheral shape around a central aperture, the peripheral shape being D-shaped with a straighter anterior side adapted to be implanted adjacent the anterior leaflet opposite a more rounded posterior side adapted to be implanted adjacent the posterior leaflet, the annuloplasty ring having an inner core and a suture-permeable interface surrounding the inner core and extending around the peripheral shape, the inner core having a size-adjustable segment adjacent just the P2-P3 regions of the posterior leaflet that is linearly or circumferentially compressible to enable constriction of the ring while the P1 region and anterior side are not size-adjustable, the system including a proximal control handle having a flexible shaft extending therefrom, a distal end of the shaft being adapted to engage an access port in the ring, and at least one tension filament extends from the control handle through the flexible shaft and access port and extends around an inner core of the ring to an anchor point, wherein an actuator on the control handle is configured to apply tension to the filament and constrict the size-adjustable segment of the inner core.

13. The system of claim 12, wherein the peripheral shape is open and defines two free ends.

14. The system of claim 12, wherein the access port is located at one of the free ends.

15. The system of claim 12, wherein the access port is located on an atrial side of the ring spaced from one of the free ends.

16. The system of claim 12, wherein the actuator is a rotatable ring.

17. The system of claim 12, wherein there are three tension filaments extending around the inner core of the ring to different anchor points, and there is one actuator on the control handle for each tension filament.

18. The system of claim 12, wherein the size-adjustable segment comprises short core members that slide relative to curved struts passing through apertures therein.

19. The system of claim 12, wherein the size-adjustable segment comprises short core members separated by linearly compressible sections.

20. The system of claim 12, wherein the core members and linearly compressible sections are both made of silicone.
